(19) United States
(12) Patent Application Publication
(10) Pub. No.: US 2025/0331860 A1
(43) Pub. Date: Oct. 30, 2025

(54) LIGATOR DEVICES, SYSTEMS, AND METHODS

(71) Applicant: Boston Scientific Scimed, Inc., Maple Grove, MN (US)

(72) Inventors: Mauro Viquez Gonzalez, Heredia (CR); Rafael Berenzon, Cartago (CR); Gabriela Gamboa Bastos, San José (CR); Esteban de la Trinidad Jiménez Sánchez, San José (CR); Marco Vinicio Arias Alpizar, San José (CR); Gonzalo Jose Sáenz Villalobos, Alajuela (CR); Oscar Segura Elizondo, San José (CR); Fernando Valerio Guerrero, Alajuela (CR); Viviana Artavia Salas, Heredia (CR); Erick Van Patten Rivera, San José (CR); Gustavo Carvajal Bonilla, Heredia (CR); Valeria Zúñiga Vargas, Austin, TX (US)

(73) Assignee: Boston Scientific Scimed, Inc., Maple Grove, MN (US)

(21) Appl. No.: 19/187,539

(22) Filed: Apr. 23, 2025

Related U.S. Application Data
(60) Provisional application No. 63/638,022, filed on Apr. 24, 2024.

(51) Int. Cl. A61B 17/12; A61B 1/00; A61B 1/018; A61B 17/00

(52) U.S. Cl. CPC A61B 17/12013; A61B 1/0008; A61B 1/018; A61B 2017/00367; A61B 2017/12018

(57) ABSTRACT

A band ligator which is mounted on an end of a delivery element, such as an elongate member, without the need to extend an actuator proximally for access by a user. Instead, after the band ligator is delivered to tissue to be ligated, a separate and independent actuation instrument may be advanced to the band ligator to actuate an actuator element thereof to deploy a ligating band.

### CROSS-REFERENCE TO RELATED APPLICATIONS

This application claims the benefit of priority under 35 U.S.C. § 119 to U.S. Provisional Application No. 63/638,022, filed Apr. 24, 2024, the entire disclosure of which is hereby incorporated by reference herein for all purposes.

### FIELD

The present disclosure relates generally to the field of ligator devices, systems, and methods. In particular, the present disclosure relates to band ligator devices, systems, and methods such as for treating varices, hemorrhoids, etc., by deploying a band around the area to be treated from a device mounted on a distal end of an elongate member. Even more particularly, the present disclosure relates to band ligator devices, systems, and methods in which the band ligator is mountable with respect to a distal end of an elongate member, the band ligator and elongate member insertable into a patient's body to deliver the band ligator to a treatment site at which ligating bands are deployed from the band ligator with the use of an instrument which is separate and independent of band ligator device and not delivered to the treatment site initially with the band ligator.

### BACKGROUND

Endoscopic variceal ligation, or endoscopic band ligation, is a procedure that uses elastic bands to treat enlarged veins, or varices, which develop in the gastrointestinal system (e.g., the esophagus, intestines, etc.). These abnormal veins typically have thin walls with blood at high pressures running through them. If not treated, the veins may rupture and cause serious bleeding. Variceal ligation may be performed during an upper gastrointestinal endoscopy. A medical scope is placed through the mouth and down to the esophagus. When varices are found, tiny elastic bands are placed around the enlarged veins in the esophagus to tie them off so they cannot bleed. Blood supply to the banded varices is substantially cut off so that the varices eventually slough off after a few days. The esophagus is much less likely to bleed after it has healed from such procedure than from a procedure which cuts the varices. Endoscopic band ligation (EBL) is the preferred endoscopic technique for the endoscopic treatment of acute esophageal variceal bleeding. EBL is generally accepted and recommended for the treatment of acute bleeding events and prophylactic treatment of esophageal variceal hemorrhage. EBL has also been used to treat nonvariceal bleeding. Additionally, EBL has been demonstrated to be a feasible and safe alternate technique for the management of iatrogenic gastric perforation, especially in cases in which closure with endoclips is difficult. Various devices for performing band ligation are configured to deliver and to release a band around tissue such as varices. Some of the most common reported failures of such devices include inability of a band to deploy; misfire or premature deployment; a band falling off after deployment; and misuse or user errors due to the setup of the device. There remains a need for improvements to band ligator devices, systems, and methods. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a band ligator, having a proximal end and a distal end, includes a band-mounting portion; one or more ligating bands mounted on said band-mounting portion; and an actuator element operably associated with said one or more ligating band to actuate said band ligator to deploy a ligating band distally from said band-mounting portion. In some aspects, the band ligator and the actuator element are configured to be deployed within a patient without an actuation instrument operably engaging the actuator element for user access to actuate the actuator element.

In some aspects, the proximal end of the band ligator is configured to be mounted on a distal end of an elongate member for delivery to tissue to be ligated with the actuator element adjacent the distal end of the elongate member and the proximal end of the band ligator.

In some aspects, the band ligator defines a proximal lumen open along the proximal end of the band ligator and the actuator element extends across the proximal lumen of the band ligator. In some aspects, the actuator element extends across the proximal lumen within the proximal lumen. In some aspects, the actuator element has a free end coupled to the proximal end of the band ligator.

In some aspects, the band ligator further includes a filament interengaging with the one or more ligating bands and extending from a distal band to a proximal band along the exterior of the band ligator to be operably coupled with the actuator element at the proximal end of the band ligator.

In some aspects, the band ligator does not include components extending proximally beyond the proximalmost end of the band ligator.

In some aspects, the actuator element does not extend proximally beyond the proximalmost end of the band ligator.

In accordance with various principles of the present disclosure, a band ligator system includes a band ligator and an actuation instrument formed separately and independently of the band ligator. In some aspects, the band ligator includes a band-mounting portion; one or more ligating bands mounted on the band-mounting portion; and an actuator element operably associated with the one or more ligating band to actuate the band ligator to deploy a ligating band distally from the band-mounting portion. In some aspects, the band ligator is configured to be mounted with respect to an elongate member for delivery to tissue to be ligated without the actuation instrument operatively associated with the band ligator; and the actuation instrument is configured to be initially operatively associated with the band ligator after the band ligator has been delivered to tissue to be ligated.

In some aspects, the band ligator is configured for use with a medical instrument, the elongate member extending distally from a control handle of the medical instrument; and when the band ligator is delivered to tissue to be ligated, mounted with respect to the elongate member of the medical instrument, the actuator element is positioned distal to the control handle of the medical instrument without the actuation instrument extending proximally to the control handle of the medical instrument. In some aspects, the actuation instrument is insertable through a lumen extending through the elongate member after the band ligator has been delivered to tissue to be ligated to operably engage the actuator element to actuate the actuator element to deploy a ligating band.

In some aspects, the actuation instrument includes an end effector configured to grasp and manipulate the actuator element. In some aspects, the end effector is in the form of one or more hooks configured to grasp and manipulate the actuator element. In some aspects, the end effector includes a plurality of jaws movable relative to one another between an open configuration and a closed configuration. In some aspects, the actuation instrument includes a handle movable to actuate the end effector to grasp the actuator element.

In some aspects, the actuation instrument includes a handle configured to move in only one direction to deploy one ligating band at a time from the band ligator.

In accordance with various principles of the present disclosure, a method is disclosed for assembling a band ligator with respect to an elongate member configured to deliver the band ligator to tissue to be ligated. In some aspects, the method includes mounting a band ligator on a distal end of the elongate member with an actuator element of the band ligator positioned adjacent the distal end of the elongate member; and delivering the band ligator to tissue to be ligated without an actuation instrument extending proximally from the actuator element for actuation of the actuator element.

In some aspects, the band ligator is assembled with respect to the elongate member such that the band ligator does not include components extending proximally beyond the proximalmost end of the band ligator. In some aspects, the elongate member is an insertion member of an endoscope, and the method further includes mounting the band ligator on the distal end of the insertion member with the actuator element extending across the working channel of the insertion member.

In some aspects, an actuation instrument is insertable through a lumen formed through the elongate member to actuate the actuator element after the band ligator has been delivered to and positioned adjacent tissue to be ligated.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within/through a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a band ligator formed in accordance with aspects of the present disclosure and positioned for mounting on a distal end of an insertion tube of an endoscope.

FIG. 2A illustrates a simplified elevational view of an example of an embodiment of a band ligator formed in accordance with various principles of the present disclosure.

FIG. 2B illustrates a cross-sectional view along line IIB-IIB in FIG. 2A.

FIG. 2C illustrates a band ligator as in FIG. 2A being actuated to deploy a band around tissue to be ligated.

FIG. 3A illustrates a simplified elevational view of an example of an embodiment of a band ligator formed in accordance with various principles of the present disclosure.

FIG. 3B illustrates a band ligator as in FIG. 3A with an actuation instrument actuating the band ligator.

FIG. 3C illustrates a band ligator as in FIG. 38 with the actuation instrument proximally retracting tissue and with a ligating band deployed from the band ligator over the retracted tissue.

FIG. 4 illustrates an example of an embodiment of a handle formed in accordance with various principles of the present disclosure and which may be used with a band ligator such as described herein.

FIG. 5A illustrates a distal perspective view of a first grip component of the handle illustrated in FIG. 4

FIG. 5B illustrates another distal perspective view of a first grip component of the handle illustrated in FIG. 4

FIG. 6 illustrates an example of an embodiment of a shaft for an actuation instrument, such as taken along line VI-VI of FIG. 4

FIG. 7 illustrates an example of an embodiment of a handle formed in accordance with various principles of the present disclosure and which may be used with a band ligator such as described herein.

FIG. 8 illustrates an example of an embodiment of an end effector which may be used with an actuation instrument formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

It will be appreciated that terms such as engage (and other grammatical forms thereof) may be used interchangeably herein with terms such as, without limitation, couple, grasp, hold, clasp, clip, anchor, attach, affix, secure, etc. (and other grammatical forms thereof), without intent to limit. As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

Band ligators disclosed herein include a tubular element (e.g., formed by a tubular wall) with respect to which one or more ligating bands are removably positioned (e.g., mounted to extend circumferentially around the tubular element). The band ligator is inserted into a patient's body (e.g., by being inserted through a natural orifice or through a surgically-created orifice, such as created with the use of a medical instrument) to be positioned inside the patient's body adjacent tissue to be ligated. For instance, in the case of treatment of esophageal varices, the band ligator may be insertable via natural orifice transluminal endoscopic surgery (NOTES), extending the tubular element into the patient's mouth and advancing the tubular element to the esophagus. An actuator element interacts with the one or more ligating bands such that movement of the element causes a ligating band to be moved off of the tubular element and circumferentially around (e.g., encircling) tissue to be ligated. In some aspects, the actuator element is operably associated with a filament interengaged with the ligating bands so that movement of the filament causes a ligating band to be deployed from the band ligator. It will be appreciated that reference may be made herein to actuation of the actuator element and/or of the band ligator, more generally, without intent to limit. In some aspects, the tissue to be ligated is suctioned into the tubular element to facilitate positioning of the ligating band circumferentially around such tissue. The ligating band may be an elastic band which contracts over and around the tissue to be ligated once positioned therearound to commence the ligation process, cutting off blood supply to the tissue encircled by the band to cause necrosis, and ultimate sloughing off of the necrosed tissue.

In accordance with various principles of the present disclosure, a band ligator is formed without an actuation instrument operably associated with the actuation instrument (to actuate the actuator element) as in prior band ligators. More particularly, the band ligator is assembled with respect to a delivery system such as the elongate member and/or an instrument including the elongate member for delivery of the band ligator to a treatment site. Such assembly does not include an actuation instrument operably engaging the actuator element of the band ligator. As such, the delivery system with the band ligator mounted thereto does not include an actuation instrument extending proximally from the band ligator to a proximal end of the delivery system for actuating (e.g., manually by a medical professional) the actuator element to deploy a ligating band. Instead, the proximalmost end of the band ligator and all components of the band ligator, as assembled with respect to the delivery system and as delivered to the treatment site, are positioned adjacent and in close proximity to the tissue to be ligated (e.g., within the patient's body) without extending to a position where a user may manipulate (e.g., manually) a component of the band ligator. A separately formed device, tool, instrument, etc. (such terms being used interchangeably herein without intent to limit), is advanceable to the band ligator (e.g., through a lumen defined through the elongate member with respect to which the band ligator is mounted) after the band ligator has been delivered to and positioned adjacent tissue to be ligated. The separately formed actuation instrument is physically distinct and separate from the band ligator, and is configured to operably engage/interact with the actuator element of the band ligator to actuate the band ligator to deploy a ligating band. The actuation instrument is deliverable to the treatment site separately (e.g., after) and apart from the band ligator. Alternatively or additionally, the actuation instrument is removable from the treatment site independently and separately from the band ligator, such as prior to removal of the band ligator from the treatment site. In some aspects, the separately formed actuation instrument is an instrument known to those of ordinary skill in the art, such as an off-the-shelf, pre-existing, commercially available instrument (i.e., not custom-made for interaction with a band ligator formed in accordance with various principles of the present disclosure). In some aspects, the separately formed actuation instrument is similar to an off-the-shelf instrument, but with modifications or adaptations to enhance use with a band ligator of the present disclosure, such as to have features facilitating use of the band ligator of the present disclosure. In some aspects, the separately formed actuation instrument may also be used grasp tissue to be ligated and to proximally pull the tissue into a proximal lumen of the band ligator to be better positioned for a ligating band to be dispensed off the band ligator and positioned circumferentially around the grasped tissue. In some aspects, the actuator element extends transversely across a lumen defined within a tubular wall of the band ligator. In some aspects, the actuation instrument is advanced to the band ligator through a lumen defined through the elongate member and generally aligned with the lumen of the band ligator across which the actuator element extends.

Various embodiments of band ligators, and associated systems and methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features in the drawings are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers increased by a multiple of 1000 and redundant description is generally omitted for the sake of brevity. Moreover, certain features in one embodiment may be used across different embodiments and are not necessarily individually labeled when appearing in different embodiments.

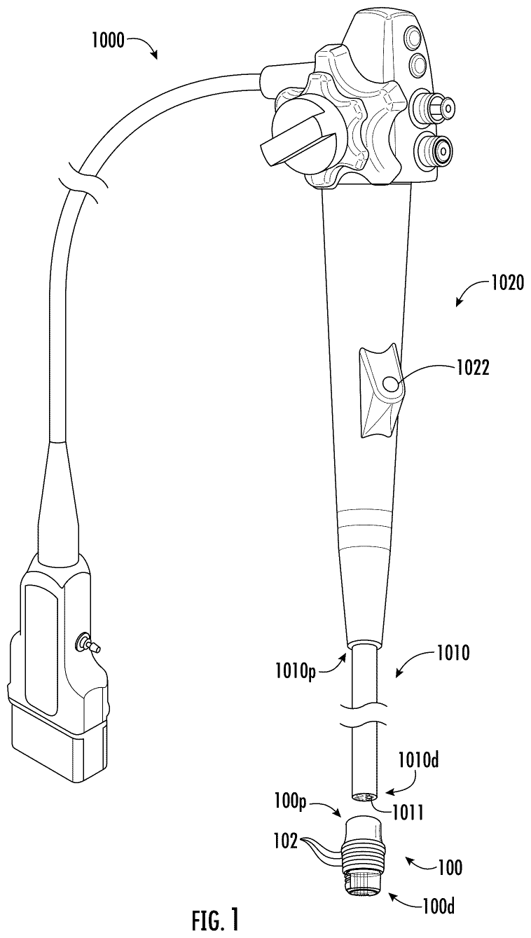

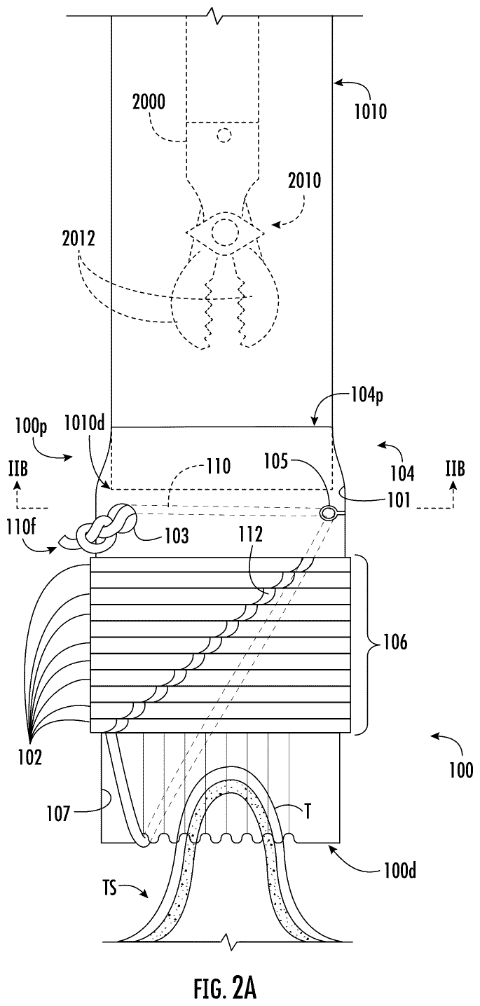

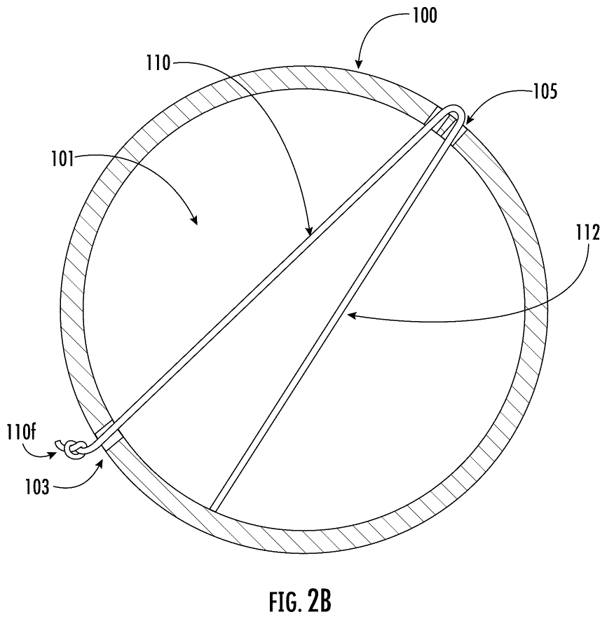

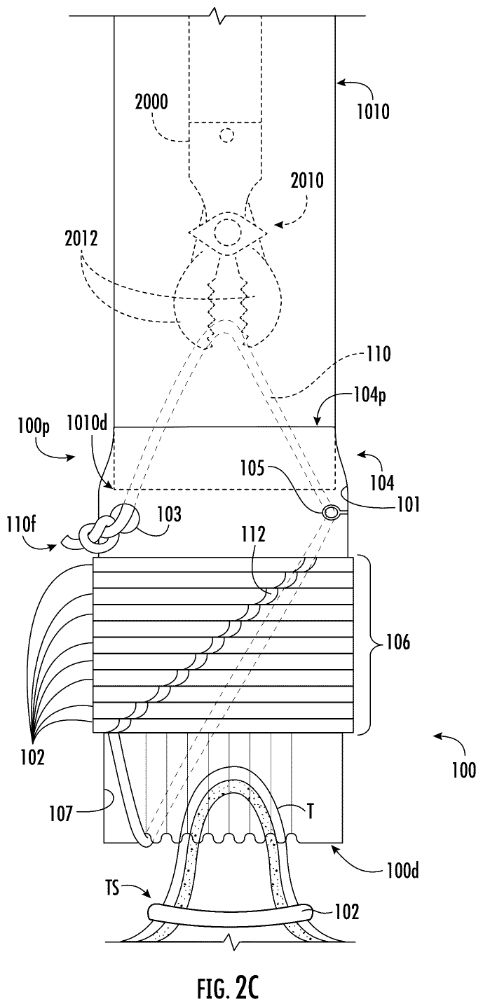

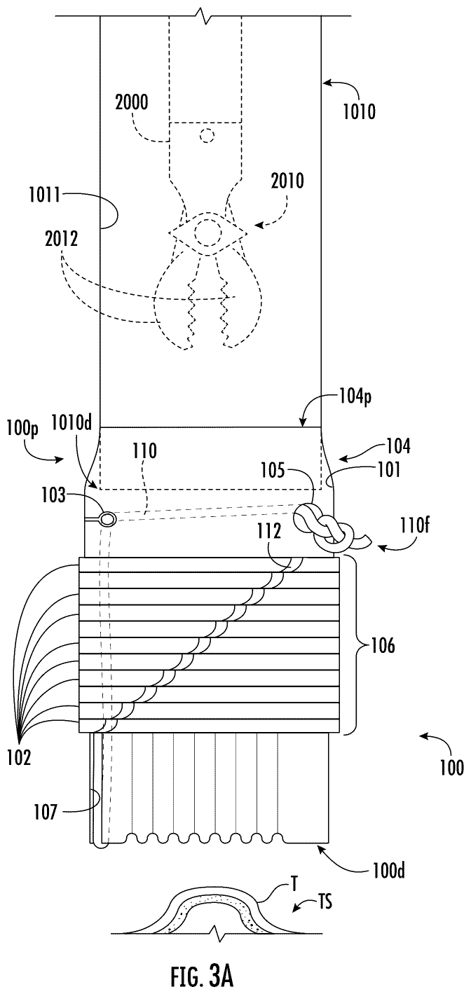

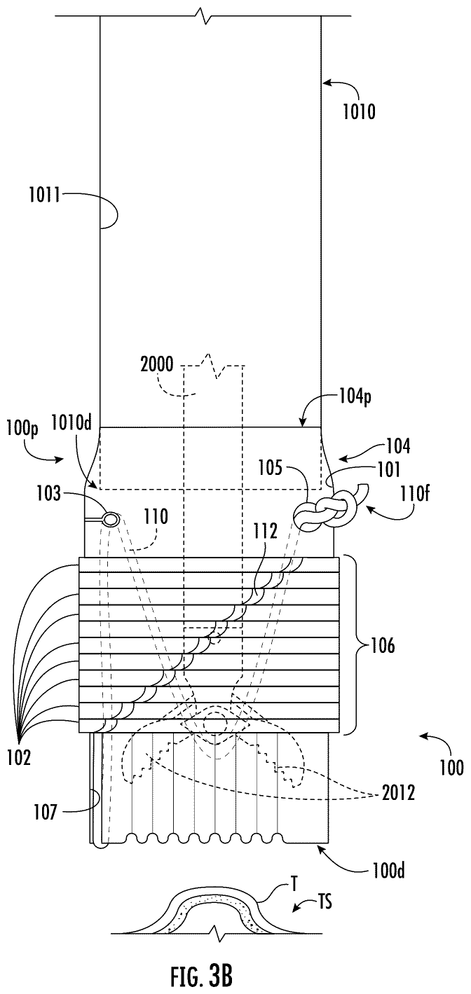

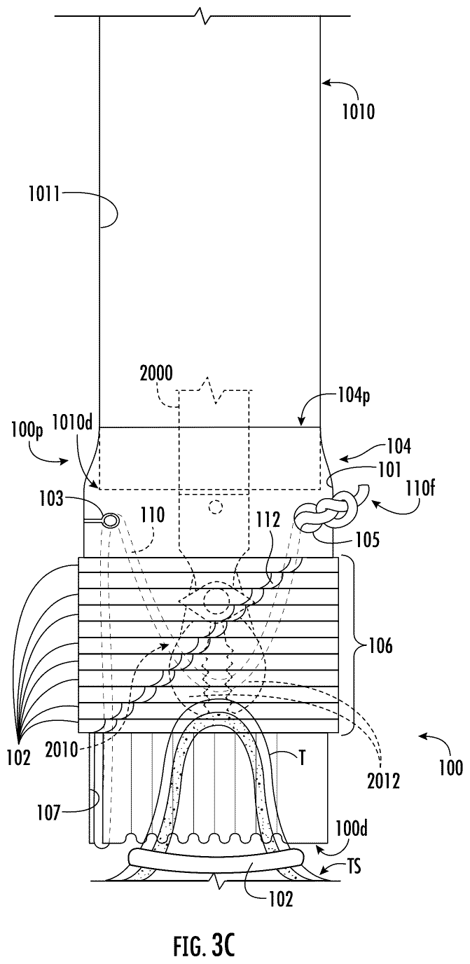

Turning now to the drawings, an example of an embodiment of a band ligator 100 formed in accordance with various principles of the present disclosure is illustrated in FIG. 1 mounted on a medical instrument 1000 (typically, although not necessarily on a distal end of the medical instrument 1000). In the illustrated example of an embodiment, the band ligator 100 is mounted with respect to (e.g., over or within) a distal end 1010d of an elongate member 1010 sized, shaped, configured, and/or dimensioned for advancement to tissue to be ligated. In some aspects, the elongate member 1010 is sized, shaped, configured, and/or dimensioned for insertion into a natural orifice of a patient. Alternatively or additionally, the elongate member 1010 may be sufficiently flexible to be able to navigate through nonlinear orifices and/or passages within the patient's body. Alternatively or additionally, the elongate member 1010 may be tubular to facilitate actuation of the band ligator 100 in a manner as described in further detail below. In the illustrated example of an embodiment of a medical instrument 1000, the elongate member 1010 extends distally from a control handle 1020 at a proximal end 1000p of the medical instrument 1000. The control handle 1020 may be used to at least control insertion of the band ligator 100 into the patient's body, and maneuvering of the position of the band ligator 100 within the patient for delivery to a treatment site TS (see, e.g., FIG. 2A and FIG. 3A). Further medical instruments may be extendable through an elongate member 1010 which is tubular, as described in further detail below. The control handle 1020 may thus include an insertion port 1022, illustrated as extending laterally with respect to a longitudinal axis Lₕ of the control handle. Although the medical instrument 1000 illustrated in FIG. 1 is a medical scope (e.g., an endoscope), with the elongate member 1010 being an insertion member thereof (the flexible elongate member of an endoscope extending distally from the control handle and which is extended into the patient), the medical instrument 1000 may be a simpler device, such as a catheter. A control handle operably associated with the elongate member 1010 is optional.

The example of an embodiment of a band ligator 100 illustrated in FIG. 1 is positioned to be mounted on a distal end 1010d of an elongate member 1010. The band ligator 100 has a proximal end 100p and a distal end 100d and a length therebetween selected such that the proximal end 100p of the band ligator 100 is distal to the proximal end 1010p of the elongate member 1010, such as distal to the control handle 1020 of the medical instrument 1000 from which the elongate member 1010 distally extends. More particularly, the proximalmost end of the band ligator 100 (i.e., the proximal free end of the band ligator 100, with no other components coupled to or otherwise a part of the band ligator 100 extending proximally from such free end) remains in close proximity to the distal end 1010d of the elongate member 1010 and is positioned spaced apart from and distal to the proximal end 1010p of the elongate member 1010 (which typically extends outside the patient's body while the band ligator 100 is positioned within the patient's body). In accordance with various principles of the present disclosure, the band ligator 100 is inserted into and navigated within a patient's body with the use of the elongate member 1010 on which the band ligator 100 is mounted, and not with its own dedicated control handle (e.g., a control handle coupled thereto, such as upon assembly of the band ligator 100 with respect to the medical instrument 1000). The band ligator 100, as initially mounted/assembled with respect to the elongate member 1010, does not have its own control handle and the proximal end 100p thereof is free of connections to a control handle dedicated to controlling operation of the band ligator 100 (e.g., dispensing of bands therefrom), such as direct connections to a control handle. As such, the actuator element 110 of the band ligator 100 may be considered to be inaccessible for actuation upon delivery to a treatment site TS (see, e.g., FIG. 2A and FIG. 3A). Operation of the band ligator 100 to deliver a ligating band 102 therefrom is controlled by a separately formed instrument that is not formed as a part of the band ligator 100 or installed with the band ligator 100 with respect to the elongate member 1010. In other words, the band ligator 100 is installed, such as mounted, coupled, assembled, or otherwise operably associated with the elongate member 1010, independently of and separately from an element which may assist with controlling operation of the band ligator 100 (e.g., to deliver a ligating band 102). A separately and independently formed actuation instrument may be delivered to the treatment site TS (e.g., after delivery of the band ligator 100 to the treatment site TS. The separate and independent actuation instrument may be used to actuate the band ligator 100, such as to cause a ligating band 102 to be released from the band ligator 100 (and, generally, over tissue to be ligated), by engaging a component of the band ligator 100 without necessarily being coupled to such component. If the actuation instrument used to actuate the band ligator 100 is coupled to a component of the band ligator 100, such coupling is typically releasable. In accordance with various principles of the present disclosure, in some aspects, engagement of an actuation instrument with a component of the band ligator 100 to actuate the band ligator 100 is achieved once the band ligator 100 has been delivered to a treatment site TS. As such, preassembly of the band ligator 100 with respect to the elongate member 1010 for delivery to the treatment site TS is minimized if not eliminated. The user simply mounts the band ligator 100 (delivered to the user already assembled for use) on the elongate member 1010 (e.g., the distal end 1010d of the elongate member 1010), without coupling or otherwise operably associating further components with the band ligator 100 prior to delivery and deployment of the band ligator 100 with respect to a treatment site TS.

To facilitate operable association of the band ligator 100 with a further instrument which may be used to actuate the band ligator 100 to deploy a ligating band 102 the elongate member 1010 may be tubular, defining a lumen 1011 therethrough. In the case of a medical instrument 1000 in the form of a medical scope, the lumen 1011 typically is the working channel of the insertion element of the scope. For the sake of convenience, and without intent to limit, the further instrument is referenced herein as an actuation instrument. As illustrated in FIG. 2A and FIG. 3A, an actuation instrument 2000 is extended through the lumen 1011 of the elongate member 1010 (with respect to which the band ligator 100 mounted) to be operably associated with the band ligator 100. More particularly, as may be appreciated with reference to FIG. 2A and FIG. 3A, at least the proximal end 100p of the band ligator 100 is tubular, defining a lumen 101 therethrough sized, shaped, configured, and/or dimensioned to receive the distal end 1010d of the elongate member 1010. It will be appreciated that the elongate member 1010 illustrated in FIG. 2 is illustrated as a simply tubular elongate member with a lumen therethrough, such as a catheter. One of ordinary skill in the art will appreciate the various proportions and modifications to be made to FIG. 2A and FIG. 3A to represent the lumen 1011 as a working channel 1011 of an insertion element of an endoscope such as illustrated in FIG. 1 It will further be appreciated that the elongate member 1010 and the actuation instrument 2000 typically are somewhat curved to place the distal end 100d of the band ligator 100 in an operable position with respect to the treatment site TS and the tissue T to be ligated, whereas the illustration is somewhat simplified in showing a substantially straight configuration. In some aspects, the proximal end 100p of the band ligator 100 includes a fitment 104 (which may also be referenced as a fitting, adaptor, etc.) fixedly coupled to the proximal end 100p of the band ligator 100, and formed from a material capable of engaging and gripping onto the exterior of the distal end 1010d of the elongate member 1010. In some aspects, the fitment 104 is formed from an expandable material and defines an inner diameter slightly smaller than the outer diameter of the elongate member 1010 so that the fitment 104 is expanded over the distal end 1010d of the elongate member 1010 and then released to contract and grip onto the distal end 1010d of the elongate member 1010. In some aspects, the fitment 104 need not be expandable. In some aspects, the material of the fitment 104 may be a plastic such as silicon and form a friction or interference fit with respect to the distal end 1010d of the elongate member 1010

One or more ligating bands 102 are mounted over and circumferentially around (e.g., encircling) the exterior of at least a portion of the band ligator 100 distal to the fitment 104. In some aspects, the ligating bands 102 are elastic and stretched into an expanded configuration to be extended, placed, positioned, etc., over the exterior of the band ligator 100. In such embodiments, the portion of the band ligator 100 over which the ligating bands 102 are mounted may be sufficiently rigid so as not to be deformed by the hoop strength of the ligating band 102 Such portion of the band ligator 100 may be referenced herein as the band-mounting portion 106 of the band ligator 100. As may be appreciated, the band-mounting portion 106 may have greater rigidity and resistance to collapsing radially inwardly than does the fitment 104 (which may be designed to be biased radially-inwardly to grip the exterior of the elongate member 1010).

In order to actuate the band ligator 100 to release a ligating band 102 the band ligator 100 includes an actuator element 110 operably associated with the ligating bands 102. In some aspects, the actuator element 110 is provided along the proximal end 100p of the band ligator 100. In some aspects, the actuator element 110 is a proximal extension of or an additional (e.g., separately formed) element coupled to a proximal end of a filament 112 (e.g., one or more wires, threads, sutures, strings, etc.). The filament 112 is interengaged with the ligating bands 102 such that manipulation of the actuator element 110 (e.g., proximal or distal movement thereof) causes a ligating band 102 to be moved distally off the band ligator 100 For instance, the filament 112 may extend along the exterior of the band ligator 100 from the proximal end of the band-mounting portion 106 to the distal end of the band-mounting portion 106, looping around ligating bands 102 in a proximal to distal direction. In such configuration, the filament 112 may then extend around the distalmost end of the band-mounting portion 106 to extend into the band ligator 100 (e.g., into the lumen 101 within the band ligator 100) to be coupled with the actuator element 110. The manner, pattern, configuration, etc., of interengagement of the actuator element 110 and/or filament 112 with the ligating bands 102 may follow any known manner, pattern, configuration, the present disclosure not being limited in this regard. For instance, the manner, pattern, configuration may follow the wrapping of a suture with respect to a plurality of ligating bands to cause release of ligating bands from a ligator described in U.S. Patent Application Publication US2020/0360022 to Schaubhut et al, published on Nov. 19, 2020. However, it will be appreciated that other configurations of operably associating the actuator element 110 with the one or more ligating bands 102 to actuate a ligating band 102 to be deployed off the distal end 100d of the band ligator 100 are within the scope and spirit of the present disclosure, the present disclosure not being limited in this regard.

Prior band ligators generally have an actuator element which requires the user to manipulate the actuator element, or another component operatively associated therewith, when mounting the band ligator with respect to the medical instrument for the actuator element 110 to be accessible for actuation after the band ligator has been delivered to a treatment site. For instance, an actuation instrument may be preassembled with the band ligator and operably associated with the actuator element and extended proximally to a position for direct user access, or must be assembled with the band ligator (i.e., the components carrying the ligating bands) by the user prior to delivery of the band ligator to the treatment site. More particularly, some prior band ligators include an actuator element having a distal end which interacts with the ligating bands (e.g., by engaging the bands, such as wrapping around or otherwise being coupled with the bands) and a proximal end extending proximally (e.g., outside the patient) or operably associated with an actuation instrument extending proximally for manual manipulation by a medical professional (e.g., by direct contact with the proximal end of the actuator element) to actuate the band ligator to release a ligating band. In other prior band ligators, the user must operably associate an actuator element/actuation instrument with the band ligator and extend the actuator element proximally for manipulation by a medical professional to actuate the actuator element to release a ligating band. The common feature in these prior band ligators is that the element which is manipulated to actuate the band ligator is assembled and coupled to the band ligator (in particular, the ligating bands) prior to delivery of the band ligator to the treatment site and/or requires assembly steps which must be performed by the user beyond simply mounting the band ligator on a medical instrument.

In contrast with prior band ligators, a band ligator 100 formed in accordance with various principles of the present disclosure does not share the generally common pre-assembly/assembly requirements of prior art band ligators, such as proximally extending an actuator coupled to the band ligator to a proximal end of the system to actuate operation of the band ligator. Instead, the actuator element 110 remains in the vicinity of/close proximity to the proximal end 100p of the band ligator 100 and does not extend proximally such as to be in the vicinity of the proximal end 1010p of the elongate member 1010 and/or to be outside the patient for manipulation by a medical professional. Instead, the actuator element 110 (including the proximalmost portion thereof) remains adjacent the proximal end 100p of the band ligator 100. In some aspects, the actuator element 110 does not extend proximally beyond the proximalmost end of the band ligator 100 other than when being actuated. In the examples of embodiments illustrated in FIG. 2A, FIG. 2B, FIG. 2C, FIG. 3A, FIG. 3B, and FIG. 3C, the proximal most end of the band ligator 100 is along the proximal end 104p of the fitment 104 However, if a fitment 104 is not provided, then the proximalmost end of the band ligator 100 is along the proximalmost end of the tubular wall of the band ligator 100 which fits over the distal end 1010d of the elongate member 1010.

In further contrast with prior band ligators, a component of the actuator element 110 of a band ligator 100 formed in accordance with various principles of the present disclosure does not extend proximally for access (e.g., direct access) by a user to be actuated to deploy a ligating band 102. Instead, the actuator element 110 and all components associated therewith remain at the distal end 1010d of the elongate member 1010 and thus spaced apart from the proximal end of the elongate member 1010 and generally inaccessible to a user when deployed. In some aspects, the proximal free end 110f of the actuator element 110 is coupled to the proximal end 100p of the band ligator 100 (e.g., to the fitment 104 or a tubular wall of the band ligator 100 with respect to which the fitment 104 proximally extends). In some aspects, the free end 110f of the actuator element 110 and the proximal end 100p of the band ligator 100 are coupled by welding (e.g., by melting plastic components thereof together) and/or a mechanical coupling. For instance, in the example of an embodiment illustrated in FIG. 2A, FIG. 2B, FIG. 2C, FIG. 3A, FIG. 38, and FIG. 3C, a portion (e.g., proximal to the free end) of the actuator element 110 passes through a hole 103 in the band ligator 100 (e.g., through the tubular wall of the band ligator 100 from which the fitment 104 extends, and/or through the fitment 104) from one side of the tubular wall of the band ligator 100 (e.g., from inside the band ligator 100) to the other side (e.g., the outside) of the tubular wall of the band ligator 100, and is secured with respect to the band ligator 100 along such other side, such as with a knot. In some aspects, the filament 112 which affects movement of the ligating bands 102 extends from the ligating band 102 (typically positioned on a band-mounting portion 106 adjacent a distal end 100d of the band ligator 100) proximally within the band-mounting portion 106 to be coupled with the actuator element 110, such as described above. In some aspects, the filament 112 has a proximal end forming the actuator element 110 (in other words, the actuator element 110 is a proximal end of and generally coextensive with the filament 112) and/or is coupled with a separately formed actuator element 110 which extends within and across the interior of the band ligator 100 (e.g., the lumen 101). As such, the actuator element 110 remains at or distal to the proximal end 100p of the band ligator 100.

Moreover, in further contrast with prior band ligators, when a band ligator 100 formed in accordance with various principles of the present disclosure is deployed at a treatment site TS, an actuation instrument 2000 is not coupled to or otherwise operably engaged or otherwise associated with the actuator element 110 to extend proximally for access (e.g., direct access) by a user when the band ligator 100 is deployed at a treatment site TS. There is no additional actuation instrument operably associated with the actuator element 110 and/or assembled, coupled, operably engaged, or otherwise associated with respect to the band ligator 100 by the user, such as upon mounting of the band ligator 100 with respect to the elongate member 1010, prior to insertion of the band ligator 100 into the patient's body. Such novel configuration of a band ligator 100 simplifies the structure of the band ligator 100 and facilitates use by the medical professional (e.g., reducing steps necessary to position and ready the band ligator 100 with the elongate member 1010). Moreover, as may be appreciated with the further descriptions below, a pre-existing, off-the-shelf medical instrument may be used as an actuation instrument, rather than a component which is specifically (e.g., custom) made for operable association with the band ligator.

In the example of an embodiment illustrated in FIG. 2A and FIG. 3A, the actuator element 110 of a band ligator 100 formed in accordance with various principles of the present disclosure extends across the proximal end 100p of the band ligator 100 (e.g., across the fitment 104) and across the lumen 101 defined therein. All components of the band ligator 100 which have been mounted on the distal end 100d of the elongate member 1010 for delivery to the treatment site TS remain in the vicinity of the distal end 1010d of the elongate member 1010, without extending proximally for access by a user for actuation of the band ligator 100. In accordance with various principles of the present disclosure, distal extension of an actuation instrument 2000 through the lumen 1011 of the elongate member 1010 over which the band ligator 100 is mounted allows the medical professional to engage and manipulate (move, advance, actuate, etc.) the actuator element 110 with the actuation instrument 2000. As described briefly above, the lumen 1011 of the elongate member 1010 is generally aligned with the lumen 101 across which the actuator element 110 extends so that distal extension of an actuation instrument 2000 through the lumen 1011 of the elongate member 1010 directs the actuation instrument 2000 into operable contact with the actuator element 110. In some aspects, the fitment 104 extends proximally beyond the actuator element 110 so that the actuator element 110 may remain distal to the distal end 1010d of the elongate member 1010 for operable contact and engagement of an actuation instrument 2000, extended through the elongate member lumen 1011, with the actuator element 110.

In accordance with various principles of the present disclosure, the actuator element 110 extends across the lumen 101 of the band ligator 100, such as illustrated in FIG. 2A, FIG. 2B, FIG. 2C, FIG. 3A, and FIG. 3B, to facilitate actuation by an actuation instrument 2000 which it not coupled thereto, but, instead, which is advanced thereto from a position (such as illustrated) proximally spaced apart from the actuator element 110. In some aspects, the band ligator 100 and/or the fitment 104 is configured to maintain the positioning of the filament 112 and the actuator element 110 so that the actuator element 110 extends across the lumen 101 For instance, the filament 112 may extend from a distal end 100d of the band ligator 100 (e.g., where the filament 112 enters the lumen 101 of the band ligator 100, as described above) towards a fixation point 105 with respect to the band ligator 100. The filament 112 extends to the actuator element 110 proximal or distal to the fixation point 105, the present disclosure not being limited in this regard, and the actuator element 110 extends from the filament 112 and the fixation point 105 across the lumen 101. In some aspects, the filament 112 may extend across the lumen 101 within the band ligator 100 to the fixation point 105, such as illustrated in FIG. 2A. Alternatively, the filament 112 may extend proximally (e.g., substantially along the interior of the wall of the band ligator 100, optionally being held or guided by any guide element known to those of ordinary skill in the art) to the fixation point 105, such as illustrated in FIG. 3A. It will be appreciated that the fixation point 105 is configured to maintain the filament 112/actuator element 110 at a point along the band ligator 100 spaced apart from (e.g., opposite, such as diametrically opposed from) the point along the band ligator 100 at which the free end 110f of the actuator element 110 is coupled (e.g., the hole 103). The filament 112 and/or the actuator element 110 is movable with respect to the fixation point 105 so that the filament 112 may be pulled proximally to release a ligating band 102 from the band ligator 100 as described above, and as described in further detail below. The fixation point 105 may be in any configuration allowing such movement of the filament 112/actuator element 110. For instance, as illustrated in FIG. 2A, the fixation point 105 is an eyelet through which a portion of the filament 112 and/or the actuator element 110 movably/translationally extends. Alternatively, the fixation point 105 may be one or more apertures through the wall of the band ligator 100, such as an even number of apertures, such as illustrated in FIG. 2B. The filament 112 and/or actuator element 110 may extend out of the lumen 101 of the band ligator 100 through an aperture, and back into the lumen 101 through another aperture, to extend therefrom to the point along the band ligator 100 at which the free end 110f of the actuator element 110 is coupled, without movement of the filament 112 and/or actuator element 110 being hindered by the apertures so that the actuator element 110 may be actuated to actuate the filament 112 to release a ligating band 102 from the band ligator 100.

In view of the above, it will be appreciated that a system in accordance with various principles of the present disclosure affords the medical professional the option to choose a preferred medical instrument (e.g., based on personal preferences, or upon specific aspects or circumstances of the procedure) as an actuation instrument, rather than being required to assemble an actuator of a band ligator with respect to the elongate member 1010. Moreover, the medical professional simply mounts the band ligator 100 with respect to the distal end 1010d of the elongate member 1010 and introduces the band ligator 100 to the treatment site TS. Once the band ligator 100 is positioned to perform band ligation, the medical professional may select an appropriate actuation instrument 2000 to introduce to the ligation system (e.g., the elongate member 1010, which is optionally a component of an endoscope, and the band ligator 100). In some aspects, a ligation system is sold as a kit with an actuation instrument 2000, but the actuation instrument 2000 need not be preassembled (e.g., operably coupled) with respect to the band ligator 100, and the band ligator 100 is delivered to the treatment site TS without an actuation instrument 2000 coupled or otherwise associated therewith. The actuation instrument 2000 may simply be operably associated with the band ligator 100 by being inserted through the lumen 1011 of the elongate member 1010 in a manner similar to typical insertion of instruments into an elongate tubular member during a medical procedure. The medical professional thus does not need to perform additional assembly steps which are specific to the band ligator and which are not typical procedural steps for the medical professional during performance of a medical procedure in general. Moreover, because the actuator element 110 extends across the lumen 101 of the band ligator 100 (e.g., of the fitment 104 of the band ligator 100), which is generally coaxial with the lumen 1011 of the elongate member 1010, distal advancement of an actuation instrument 2000 through the lumen 1011 of the elongate member 1010 should bring the end effector 2010 of the actuation instrument 2000 into operable engagement with the actuator element 110 with little to no effort. It will be appreciated that if the elongate member 1010 is an insertion tube of an endoscope, then the band ligator 100 should be positioned with respect to the distal end 1010d of the elongate member 1010 with the actuator element 110 positioned to extend across the working channel 1101 of the elongate member 1010 through which the actuation instrument 2000 is advanced.

In accordance with various principles of the present disclosure, the tissue T to be ligated, which is positioned at the treatment site TS to which the band ligator 100 is delivered, may be extended proximally to facilitate positioning of the ligating band 102 therearound upon deployment from the band ligator 100. In some aspects, the tissue T to be ligated is pulled proximally into the distal end of the lumen 101 of the band ligator 100. In the example of an embodiment illustrated in FIG. 2A, FIG. 2B, FIG. 2C, FIG. 3A, FIG. 3B, and FIG. 3C, the band ligator 100 is generally tubular (e.g., formed with a generally tubular wall). In some aspects, the lumen 101 through the proximal end 100p of the band ligator 100 extends substantially continuously through the band-mounting portion 106 to the distal end 100d of the band ligator 100. However, in other configurations, the lumen 101 may not extend continuously within the band ligator 100. For instance, a separate distally-opening lumen 107 may be formed within the distal end 100d of the band ligator 100. In such embodiment, the lumens 101, 107 may be in sufficient communication with one another to allow the filament 112 to pass through the distally-opening lumen 107 and into and through the lumen 101. The distal opening of the lumen 101 (or distally-opening lumen 107) is sized, shaped, configured, and/or dimensioned for tissue T to be ligated at the treatment site TS to be pulled into the distally-opening lumen 101 (or the distally-opening lumen 107). The tissue T to be ligated may be pulled into the distally-opening lumen 107 by suction (e.g., a vacuum applied through the band ligator 100, such as via the elongate member 1010) or otherwise (e.g., grasped and pulled, such as described in further detail below). Thus, the tissue T to be ligated is properly positioned for the ligating band 102 to be dispensed therearound and released to ligate the tissue T.

The ligating band 102 is deployed by manipulating the actuator element 110, such as moving the actuator element 110 distally or proximally with respect to the band-mounting portion 106. The filament 112 operably engaging the ligating bands 102 is thereby moved with respect to the band-mounting portion 106 and moves the ligating bands 102 as well. The filament 112 and actuator element 110 are operably engaged by the actuation instrument 2000 so that the movement the medical professional performs with the actuation instrument 2000 causes the actuator element 110 to move in a direction which moves the filament 112 to deploy the ligating bands 102 distally off the band-mounting portion 106.

In accordance with various principles of the present disclosure, the actuation instrument 2000 has an end effector 2010 configured to operably engage the actuator element 110 to manipulate the actuator element 110 to cause a ligating band 102 to be deployed and released from the band ligator 100. In the example of an embodiment of an actuation instrument 2000 illustrated in FIG. 2A and FIG. 2C, the end effector 2010 is a forceps, although other configurations are within the scope of the present disclosure. The illustrated end effector 2010 has two or more jaws 2012 movable towards and away from each other to allow the actuator element 110 to be positioned between the jaws 2012 and for the jaws 2012 to be moved into a position capturing the actuator element 110 therebetween. It will be appreciated that the jaws 2012 may be distally advanced to the band ligator 100 in a closed configuration (with the jaws 2012 adjacent, and optionally contacting, each other), and moved to an open configuration (spaced apart from each other) once positioned sufficiently adjacent the actuator element 110 to allow the jaws 2012 to be moved to an open configuration to grasp the actuator element 110. The end effector 2010 is advanced distally to engage the actuator element 110 and then actuated (e.g., via a proximal control handle, such as by any manner known to those of ordinary skill in the art) to grasp the actuator element 110 (e.g., to move the jaws 2012 together into a closed configuration grasping or enclosing the actuator element 110 therebetween). In the example of an embodiment illustrated in FIG. 2A and FIG. 2C, once the actuator element 110 has been securely grasped by the end effector 2010 (e.g., with the actuator element 110 captured by the jaws 2012), the actuation instrument 2000 may be proximally withdrawn, as illustrated in FIG. 2C, to move the actuator element 110 to cause the ligating bands 102 to be deployed and released from the band ligator 100. The actuator element 110 and filament 112 are arranged with respect to each other and the ligating bands 102 such that the proximal movement of the actuator element 110 actuates the filament 112 to distally deploy the ligating bands 102 off the band-mounting portion 106 of the band ligator 100 to be positioned circumferentially around the tissue T to be ligated. With the tissue T to be ligated at least partially extended proximally into the distally-opening lumen 107 of the band ligator 100, actuation of the band ligator 100 to distally deploy a ligating band 102 should result in the ligating band 102 being deployed in a position to surround/encircle the tissue T to be ligated. Retraction of the ligating band 102 circumferentially around the periphery of the tissue T to be ligated commences the ligation process. The ligating band 102 may be elastic and in an extended configuration when positioned on the band-mounting portion 106 of the band ligator 100, and, when deployed, may return, on its own without further actuation, to a contracted configuration sufficiently contracted to tighten around the tissue T to ligate the tissue T. Alternatively or additionally, the ligating band 102 may be actuated to contract around the tissue T to be ligated in any manner known to those of ordinary skill in the art. It will be appreciated that the path the filament 112 follows within the band ligator 100 need not be as illustrated in FIG. 2A, FIG. 2B, and FIG. 2C, but, instead, may be a path such as illustrated in FIG. 3A and FIG. 3B.

Although the actuator element 110 is illustrated in FIG. 2A, FIG. 2B, and FIG. 2C as being moved proximally to actuate deployment of one or more ligating bands 102 other movements of the actuator element 110 may cause actuation of the band ligator 100 to deploy one or more ligating bands 102 For instance, in the example of an embodiment illustrated in FIG. 3A, FIG. 3B, and FIG. 3C, distal movement of the actuator element 110 actuates deployment of a ligating band 102. More particularly, an actuation instrument 2000 with an end effector 2010 similar to the end effector 2010 illustrated in FIG. 2A and FIG. 2C, is illustrated actuating the actuator element 110 by moving the actuator element 110 distally to cause the filament 112 to cause a ligating band 102 to be deployed from the band ligator 100. The jaws 2012 may be further distally advanced in the open configuration or in a closed configuration. In some aspects, the end effector 2010 may be further distally advanced to engage the tissue T to be ligated, such as illustrated in FIG. 3B. The actuator element 110 and filament 112 may be arranged to deploy a ligating band 102 once the actuator element 110 has been moved to the distal end 100d of the band ligator 100 so that the ligating band 102 is moved distally off the band-mounting portion 106 to be deployed over and circumferentially around the tissue T to be ligated as the end effector 2010 grasps the tissue T to be ligated. It will be appreciated that the path the filament 112 follows within the band ligator 100 need not be as illustrated in FIG. 3A and FIG. 3B, but, instead, may be a path such as illustrated in FIG. 2A, FIG. 2B, and FIG. 2C.

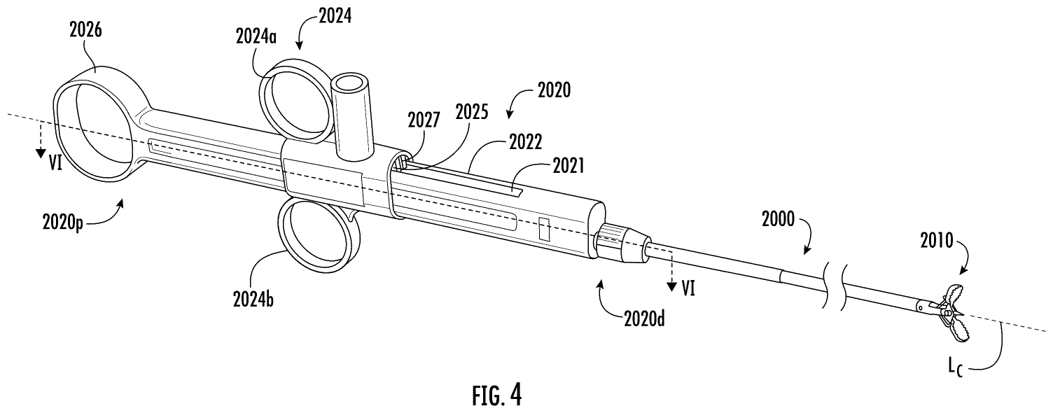

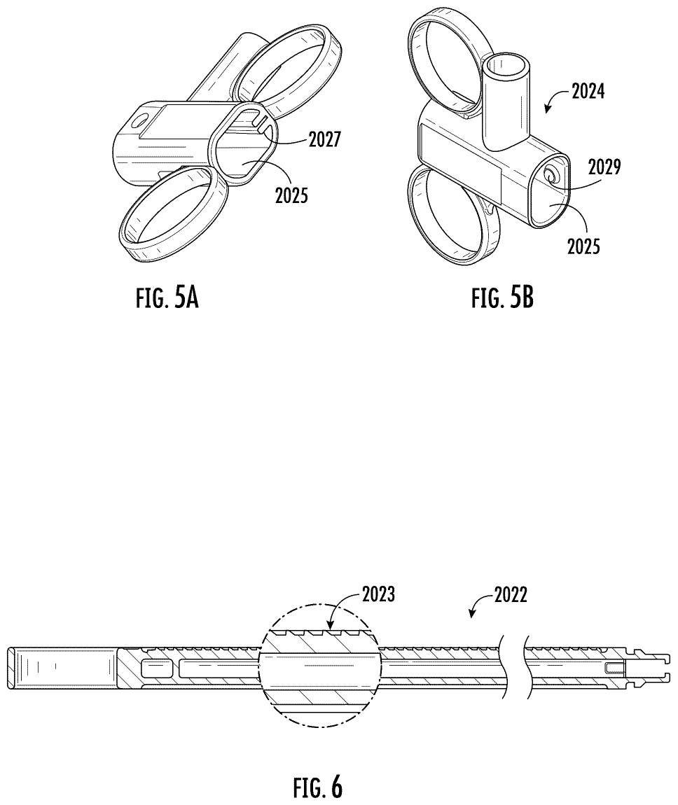

As noted above, the actuation instrument 2000 may include an actuator control handle which may be grasped by a medical professional to manipulate/control the position, orientation, etc., of the actuation instrument 2000, and optionally to control the operation of the end effector 2010 An example of an embodiment of an actuator control handle 2020 of an actuation instrument 2000 such as illustrated in FIG. 2 is illustrated in FIG. 4 with the end effector 2010 extending distally from the distal end 2020d of the actuator control handle 2020. The example of an embodiment of an actuator control handle 2020 illustrated in FIG. 4 includes a shaft 2022 along which a first grip component 2024 is axially translatable along the control handle longitudinal axis L_{c}. In some aspects, the first grip component 2024 includes one or more finger loops 2024a, 2024b through which a medical professional may insert a finger to control translation of the first grip component 2024 along the shaft 2022. A second grip component 2026 (e.g., a finger loop such as a thumb ring) may be provided along (formed integrally with, or formed separately and coupled to) the proximal end 2020p of the actuator control handle 2020. The illustrated example of an embodiment of a first grip component 2024 defines a channel 2025 for slidably receiving the shaft 2022 so that the first grip component 2024 may translate over the shaft 2022. In some aspects, the shaft 2022 includes a slide channel 2021 and the first grip component channel 2025 includes a slide projection 2027 (e.g., spline, rail, etc.) configured to extend within the slide channel 2021, as illustrated in FIG. 4, and in further detail in FIG. 5A, to facilitate translation of the first grip component 2024 along the shaft 2022.

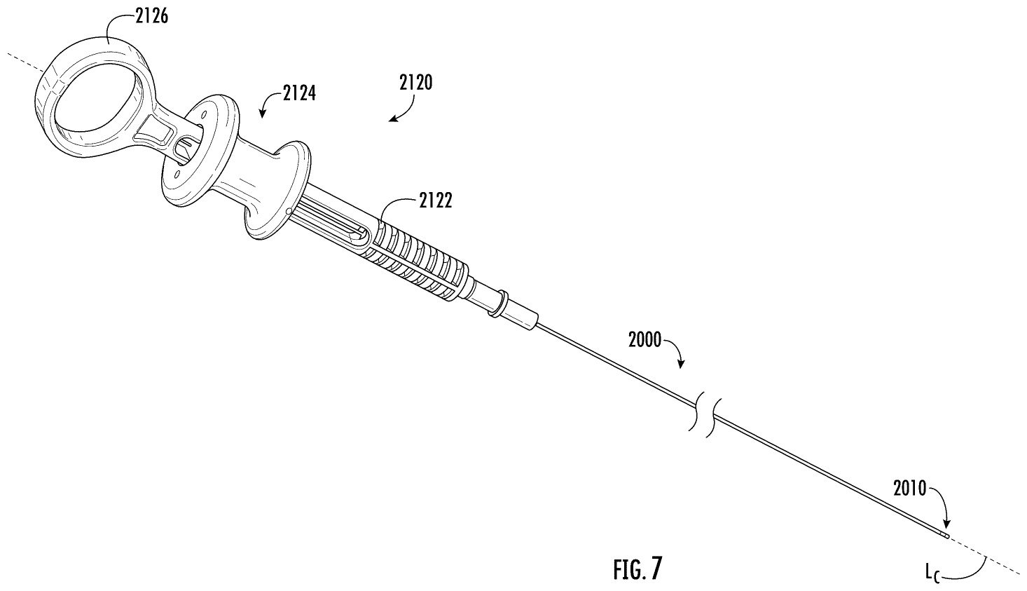

In accordance with various principles of the present disclosure, it may be desirable for the ligating system disclosed herein to limit the actuation instrument 2000 from deploying more than one ligating band 102 at a time. Alternatively or additionally, it may be desirable for the ligating system disclosed herein to indicate the number of ligating bands 102 which have been deployed by the system. As illustrated in FIG. 6, the shaft 2022 of the actuation instrument 2000 may include a set of projections 2023 (e.g., teeth, serrations, etc., such terms being usable interchangeably herein without intent to limit), and the first grip component 2024 may include a corresponding grip projection 2029 within the channel 2025, as illustrated in FIG. 5B, positioned to engage and ride along the set of projections 2023 Although the set of projections 2023 are illustrated positioned along a sidewall, the present disclosure need not be limited to such location. The same applies to the position of the grip projection 2029 so long as it is positioned to engage and ride along the set of projections 2023. In some aspects, the set of projections 2023 are spaced apart to correspond with deployment of a ligating band 102 As the first grip component 2024 is moved proximally to deploy a ligating band 102 from the band ligator 100 (such as described above with reference to FIG. 2), the grip projection 2029 moves over one of the set of projections 2023. Movement of the grip projection 2029 with respect to one of the set of projections 2023 may provide tactile and/or audible feedback to the medical professional, such as to indicate deployment of a ligating band 102. Alternatively or additionally, the next proximal of the set of projections 2023 sufficiently inhibits proximal movement of the grip projection 2029 so that the medical professional is alerted (e.g., by the resulting resistance of the first grip component 2024 to proximal movement) that further movement will release another ligating band 102. In some aspects, indicia (such as numbers) may be provided along the shaft 2022 to indicate the number of ligating band 102 deployed from the band ligator 100. It will be appreciated that the set of projections 2023 may inhibit/prevent the first grip component 2024 from distal movements.

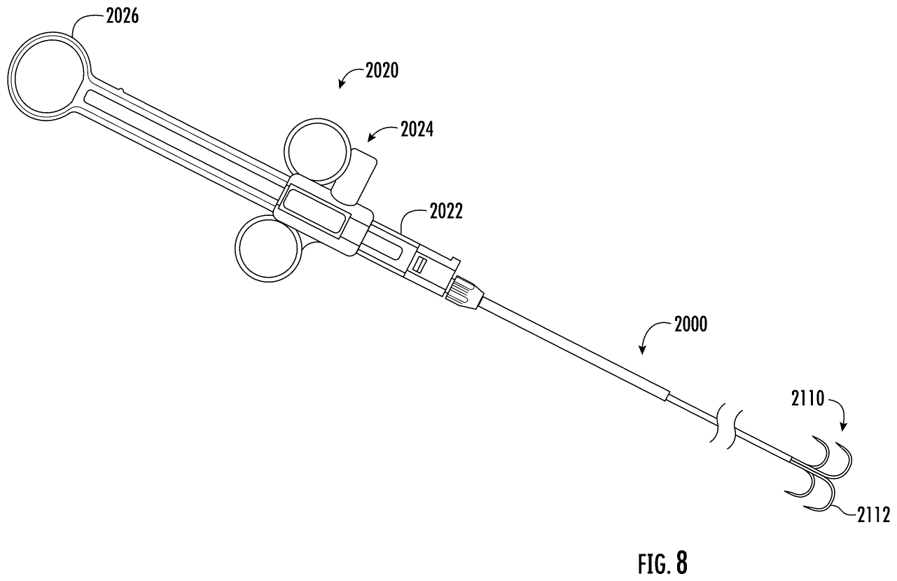

In the example of an embodiment of an actuation instrument 2000 and band ligator 100 illustrated in FIG. 3A, FIG. 3B, and FIG. 3C, relatively uninhibited proximal and distal movements of grip portion of an actuator control handle for an actuation instrument 2000 may be desirable to allow for control of movements of the end effector 2010. More particularly, end effectors 2010 typically are actuated by distal and/or proximal translation of a grip component along a shaft of a control handle. An example of an embodiment of an actuator control handle 2120 which may be used to actuate the actuation instrument 2000 and end effector 2010 of the example of an embodiment of a ligating system illustrated in FIG. 3A, FIG. 3B, and FIG. 3C is illustrated in FIG. 7. Similar to the actuator control handle 2020 illustrated in FIG. 4, the actuator control handle 2120 illustrated in FIG. 7 includes a shaft 2122 a first grip component 2124, and a second grip component 2126. However, unlike the actuator control handle 2020 illustrated in FIG. 4, the actuator control handle 2120 illustrated in FIG. 7 does not include structures inhibiting or otherwise affecting movement (e.g., longitudinal translation proximally and/or distally along the control handle longitudinal axis L_{c}) of the first grip component 2124. The ability of the first grip component 2124 to move freely, such as in both distal and proximal directions, allows the first grip component 2124 to control movement of jaws 2012 with respect to one another. For instance, as described above with reference to FIG. 3A, FIG. 3B, and FIG. 3C, the actuation instrument 2000 may be distally advanced to the target site TS with the jaws 2012 in a closed configuration, the actuator control handle 2120 may be actuated (e.g., by axially translating the first grip component 2124 relative to the shaft 2122) to move the jaws 2012 to an open configuration to grasp the actuator element 110 and/or the tissue T to be ligated, and then actuated again to move the jaws 2012 to a closed configuration again (e.g., by being axially translated in a direction opposite the direction in which the first grip component 2124 was moved to open the jaws 2012), and then actuated yet again to move the jaws 2012 to an open position to release the tissue T. Further opening and closing of the jaws 2012 may be required for further manipulation of the actuator element 110 or otherwise.

As noted above, the end effector of an actuation instrument 2000 used to actuate a band ligator 100 formed in accordance with various principles of the present disclosure need not be in the form of a forceps with jaws. Various other configurations are within the scope of the present disclosure. Another example of an end effector 2110 which may be used to operably engage an actuator element 110 of a band ligator 100 formed in accordance with various principles of the present disclosure is illustrated in FIG. 8. The example of an embodiment of an end effector 2010 illustrated in FIG. 8 is in the form of one or more hooks 2112. The hooks 2112 may be distally advanced into operable engagement with the actuator element 110 of a band ligator 100 such as illustrated in FIG. 2A, FIG. 2C, FIG. 3A, and/or FIG. 3B, and as described above. The hooks 2112 are configured to facilitate engagement with the actuator element 110 such that movement of the hooks 2112 once operably engaged with the actuator element 110, will cause the actuator element 110 to move and actuate the band ligator 100 to deploy a ligating band 102. In the illustrated example of an embodiment, the actuation instrument 2000 includes an actuator control handle 2020 similar to the actuator control handle 2020 described above with reference to FIG. 4. Operation and use of the actuator control handle 2020 illustrated in FIG. 8 may be substantially the same as operation and use of the actuator control handle 2020 described above with reference to FIG. 4. Accordingly, for the sake of brevity, reference is made to the above description of the actuator control handle 2020 of FIG. 4 as applicable to the actuator control handle 2020 of FIG. 8, without intent to limit.

As may be appreciated, because the band ligator 100 described herein is not assembled with an actuation instrument operably associated therewith prior to delivery of the band ligator 100 to tissue T to be ligated, once ligation of tissue has been completed, the band ligator 100 simply needs to be removed from the elongate member 1010 without further steps (e.g., removal of an actuation instrument extending along the elongate member 1010 from the band ligator 100 to the actuator control handle 1020 of the medical instrument 1000. If a reusable medical instrument 1000 (e.g., reusable endoscope) is used, the band ligator 100 may thus be readily removed therefrom for sterilization of the medical instrument 1000.

It will be appreciated that the present disclosure is not limited by a particular manner delivering a band ligator to a treatment site (e.g., percutaneously, endoscopically, laparoscopically, or combinations thereof). The medical devices described herein may include a variety of medical devices for navigating body lumens, including, for example, catheters, ureteroscopes, bronchoscopes, colonoscopes, arthroscopes, cystoscopes, hysteroscopes, and the like. Such devices may be disposable (i.e., intended for a single use) or sterilizable and reusable. It will be appreciated that various steps described herein may be performed within or outside of the patient's body, and do not necessarily occur in a specific order and/or timing.

The foregoing discussion has broad application and has been presented for purposes of illustration and description and is not intended to limit the disclosure to the form or forms disclosed herein. It is to be understood by one of ordinary skill in the art that the present discussion is a description of illustrative examples of embodiments only, and is not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It will thus be understood that various additions, modifications, and substitutions may be made to embodiments disclosed herein without departing from the concept, spirit, and scope of the present disclosure. In particular, it should be apparent to those of ordinary skill in the art that principles of the present disclosure may be embodied in other forms, structures, arrangements, proportions, and with other elements, materials, and components, and other that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. For example, various features of the disclosure are grouped together in one or more aspects, embodiments, or configurations for the purpose of streamlining the disclosure. Alternatively or additionally, elements shown as integrally formed may be constructed of multiple parts, and elements shown as multiple parts may be integrally formed, the operation of elements may be reversed or otherwise varied, the size or dimensions of the elements may be varied. Various further benefits of the various aspects, features, components, and structures of a band ligator and band ligator system and associated method such as described above, in addition to those discussed above, may be appreciated by those of ordinary skill in the art. Therefore, the present invention is not limited to only the embodiments specifically described herein, and all substitutes and modifications apparent to those skilled in the art are deemed to be within the spirit, scope, and concept of the disclosure as defined by the appended claims. The scope of the claimed subject matter is indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A band ligator having a proximal end and a distal end, said band ligator comprising:
a band-mounting portion;
one or more ligating bands mounted on said band-mounting portion; and
an actuator element operably associated with said one or more ligating band to actuate said band ligator to deploy a ligating band distally from said band-mounting portion;
wherein said band ligator and said actuator element are configured to be deployed within a patient without an actuation instrument operably engaging said actuator element for user access to actuate said actuator element.

2. The band ligator of claim 1, wherein the proximal end of said band ligator is configured to be mounted on a distal end of an elongate member for delivery to tissue to be ligated with said actuator element adjacent the distal end of the elongate member and the proximal end of said band ligator.

3. The band ligator of claim 1, wherein said band ligator defines a proximal lumen open along the proximal end of said band ligator and said actuator element extends across the proximal lumen of said band ligator.

4. The band ligator of claim 3, wherein said actuator element extends across said proximal lumen within said proximal lumen.

5. The band ligator of claim 4, wherein said actuator element has a free end coupled to the proximal end of said band ligator.

6. The band ligator of claim 1, further comprising a filament interengaging with said one or more ligating bands and extending from a distal band to a proximal band along the exterior of said band ligator to be operably coupled with said actuator element at the proximal end of said band ligator.

7. The band ligator of claim 1, wherein said band ligator does not include components extending proximally beyond the proximalmost end of said band ligator.

8. The band ligator of claim 1, wherein said actuator element does not extend proximally beyond the proximalmost end of said band ligator.

9. A band ligator system comprising:
a band ligator comprising:
a band-mounting portion;
one or more ligating bands mounted on said band-mounting portion; and
an actuator element operably associated with said one or more ligating band to actuate said band ligator to deploy a ligating band distally from said band-mounting portion; and
an actuation instrument formed separately and independently of said band ligator;
wherein:
said band ligator is configured to be mounted with respect to an elongate member for delivery to tissue to be ligated without the actuation instrument operatively associated with said band ligator; and
said actuation instrument is configured to be initially operatively associated with said band ligator after said band ligator has been delivered to tissue to be ligated.

10. The system of claim 9, wherein:
said band ligator is configured for use with a medical instrument, the elongate member extending distally from a control handle of the medical instrument; and
when said band ligator is delivered to tissue to be ligated, mounted with respect to the elongate member of the medical instrument, said actuator element is positioned distal to the control handle of the medical instrument without said actuation instrument extending proximally to the control handle of the medical instrument.

11. The system of claim 10, wherein said actuation instrument is insertable through a lumen extending through the elongate member after said band ligator has been delivered to tissue to be ligated to operably engage said actuator element to actuate said actuator element to deploy a ligating band.

12. The system of claim 9, wherein said actuation instrument includes an end effector configured to grasp and manipulate said actuator element.

13. The system of claim 12, wherein said end effector is in the form of one or more hooks configured to grasp and manipulate said actuator element.

14. The system of claim 12, wherein said end effector includes a plurality of jaws movable relative to one another between an open configuration and a closed configuration.

15. The system of claim 12, wherein said actuation instrument includes a handle movable to actuate said end effector to grasp said actuator element.

16. The system of claim 9, wherein said actuation instrument includes a handle configured to move in only one direction to deploy one ligating band at a time from said band ligator.

17. A method of assembling a band ligator with respect to an elongate member configured to deliver the band ligator to tissue to be ligated, said method comprising:
mounting a band ligator on a distal end of the elongate member with an actuator element of the band ligator positioned adjacent the distal end of the elongate member; and
delivering the band ligator to tissue to be ligated without an actuation instrument extending proximally from the actuator element for actuation of the actuator element.

18. The method of claim 17, wherein the band ligator is assembled with respect to the elongate member such that the band ligator does not include components extending proximally beyond the proximalmost end of the band ligator.

19. The method of claim 17, wherein the elongate member is an insertion member of an endoscope, said method further comprising mounting the band ligator on the distal end of the insertion member with the actuator element extending across the working channel of the insertion member.

20. The method of claim 17, wherein an actuation instrument is insertable through a lumen formed through the elongate member to actuate the actuator element after the band ligator has been delivered to and positioned adjacent tissue to be ligated.
